(19) United States
(12) Patent Application Publication
(10) Pub. No.: US 2024/0348711 A1
(43) Pub. Date: Oct. 17, 2024

(54) ACCESESSORY MOUNT, ACCESSORY ASSEMBLY, AND ACCESSORY KIT FOR A PORTABLE ELECTRONIC DEVICE

(71) Applicant: ConfiVox Inc., Toronto (CA)

(72) Inventor: John David Lee, Toronto (CA)

(21) Appl. No.: 18/633,684

(22) Filed: Apr. 12, 2024

Related U.S. Application Data
(60) Provisional application No. 63/459,317, filed on Apr. 14, 2023.

(51) Int. Cl. H04M 1/04; G06F 1/16

(52) U.S. Cl. CPC H04M 1/04; G06F 1/1628

(57) ABSTRACT

An accessory mount for a portable electronic device includes a mount body having longitudinally opposed first and second sides and laterally opposed third and fourth sides and a device fastener affixed to the mount body for releasably securing the accessory mount to the portable electronic device. First, second, third and fourth accessory attachment members extend respectively from and about the first, second, third and fourth side of the mount body. The first and second accessory attachment members are spaced longitudinally apart by a first distance for slidable engagement with a first electronic device accessory. The third and fourth accessory attachment members are spaced laterally apart by a second distance for slidable engagement with a second electronic device accessory. The first distance is greater than the second distance.

### CROSS-REFERENCE TO RLEATED APPLICATIONS

This application claims the benefit of U.S. Provisional Application No. 63/459,317 filed on Apr. 14, 2023, which is incorporated by reference herein in its entirety.

### FIELD

This disclosure relates generally to accessories for portable electronic devices, such as phones, tablets, laptops, etc. In one aspect, the teaching disclosed herein is directed to an accessory mount that includes accessory mounting features that promote user customization. In another aspect, the teaching disclosed herein is directed to an accessory assembly for a portable electronic device that can be used to support the portable electronic device in a handsfree viewing position.

### BACKGROUND

U.S. Pat. No. 11,483,703 (Lee) discloses a portable device accessory including an attachment mechanism, a hinge member, and a privacy shield. The attachment mechanism is for attaching the accessory to a rear face of the portable device. The rear face is opposite a front face of the portable device. The front face includes the screen of the portable device. The hinge member is pivotably attached to the attachment mechanism. The privacy shield is pivotably attached to the hinge member and movable between a storage position and a blocking position. When the privacy shield is in the blocking position, the privacy shield extends over the front face of the portable device and blocks at least a portion of the screen. When the privacy shield is in the storage position, the privacy shield extends over the rear face of the portable device and is positioned proximate to the attachment mechanism.

U.S. Patent Publication No. 2002/0180694 (Isaacson) discloses a cover for use with a portable display device. The cover has a lid which is rotatable between a closed position, through intermediate open positions, to a completely open position. A movable hinge is attached to the lid, such that the lid can rotate about the movable hinge. A fixed hinge is secured to a main body of the portable display device. The movable hinge is mounted to the fixed hinge to allow the movable hinge to be moved in an arcuate motion about the fixed hinge. Positive stop members are provided on the movable hinge and fixed hinge, whereby as the lid is rotated, the positive stop members define the intermediate open positions which position the lid at an angle relative to the main body, thereby allowing the lid to act as a stand for the portable display device. A connecting arm extends between and is pivotally mounted to the movable hinge and the fixed hinge. The stop members are in the form of projections and recesses, whereby as the lid is rotated, the intermediate open positions are defined by the projections being received and maintained in the recesses by resilient spring forces.

### SUMMARY

The following summary is intended to introduce the reader to various aspects of the applicant's teachings, but not to define any invention.

In one aspect, an accessory mount for a portable electronic device is disclosed. The accessory mount includes a mount body having longitudinally opposed first and second sides and laterally opposed third and fourth sides and a device fastener affixed to the mount body for releasably securing the accessory mount to the portable electronic device. The accessory mount further includes a first accessory attachment member extending from and about the first side of the mount body, a second accessory attachment member extending from and about the second side of the mount body, a third accessory attachment member extending from and about the third side of the mount body, and a fourth accessory attachment member extending from and about the fourth side of the mount body. The first and second accessory attachment members are spaced longitudinally apart by a first distance for slidable engagement with a first electronic device accessory. The third and fourth accessory attachment members are spaced laterally apart by a second distance for slidable engagement with a second electronic device accessory, and the first distance is greater than the second distance.

In some examples, the device fastener includes a sidewall extending generally perpendicularly from a periphery of the mount body and a device retaining ledge extending inboard from an upper edge of the sidewall.

In some examples, at least a portion of the mount body, the sidewall, and the retaining ledge define a recess for receiving a peripheral portion of the electronic device.

In some examples, each of the first, second, third and fourth accessory attachment members extends outboard of the sidewall.

In some examples, each of the first, second, third and fourth accessory attachment members extends between 1 mm and 5 mm outboard of the sidewall.

In some examples, the device fastener comprises a plurality of fastener segments. Each fastener segment is spaced apart from an adjacent one of the plurality of fastener segments along the periphery of the mount body. Each fastener segment includes a portion of the sidewall and a portion of the device retaining ledge.

In some examples, each of the first, second, third and fourth accessory attachment members extends respectively along a full length of the first, second, third, and fourth sides of the mount body.

In some examples, the accessory mount further includes an uninterrupted peripheral attachment lip extending about a perimeter of the mount body. The peripheral attachment lip includes the first, second, third and fourth attachment members.

In some examples, the mount body includes a front surface and a rear surface opposite the front surface. The front surface of the mount body at least partially overlies a rear surface of the portable electronic device when the accessory mount is secured to the portable electronic device.

In some examples, the first, second, third and fourth accessory attachment members are flush with the rear surface of the mount body.

In some examples, the mount body includes at least one device accessibility aperture. When the accessory mount is secured to the portable electronic device, the device accessibility aperture overlies one or more components of the portable electronic device to permit unimpeded function of those one or more components.

In some examples, the attachment members, the mount body, and the at least one device fastener are of unitary construction.

In some examples, the mount body includes a mount magnet for magnetically engaging a device magnet of the portable electronic device to strengthen securement between the accessory mount and the portable electronic device.

In another aspect, an accessory kit for a portable electronic device is disclosed. The accessory kit includes an accessory mount and a first device accessory slidably engageable with the accessory mount. The accessory mount includes a mount body having longitudinally opposed first and second sides and laterally opposed third and fourth sides, and at least one device fastener affixed to the mount body for releasably securing the accessory mount to the portable electronic device. The accessory mount further includes a first accessory attachment member extending from and about the first side of the mount body, a second accessory attachment member extending from and about the second side of the mount body, a third accessory attachment member extending from and about the third side of the mount body, and a fourth accessory attachment member extending from and about the fourth side of the mount body. The first and second accessory attachment members are spaced longitudinally apart by a first distance. The third and fourth accessory attachment members are spaced laterally apart by a second distance. The first distance is greater than the second distance. The first device accessory is slidably engageable with the accessory mount via the third and fourth accessory attachment members.

In some examples, the first device accessory includes a pair of opposed mounting members spaced laterally apart by the second distance. Each mounting member of the first device accessory is configured to slidably engage a corresponding one of the third and fourth accessory attachment members of the accessory mount.

In some examples, the first device accessory includes a pair of laterally spaced apart first accessory sidewalls. Each mounting member of the first device accessory is provided as a track extending along a respective one of the first accessory sidewalls for receiving one of the third and fourth accessory attachment members of the accessory mount.

In some examples, the first device accessory includes a first segment and a second segment pivotably connected to a lateral edge of the first segment. The first device accessory is movable between a storage position, in which the first and second segments of the first device accessory extend generally parallel to one another, and at least a device standing position, in which the first segment of the first device accessory is inclined relative to the second segment of the first device accessory by a stand angle.

In some examples, the stand angle is between 40 and 100 degrees.

In some examples, each first accessory sidewall has a first sidewall portion provided on the first segment and a second sidewall portion provided on the second segment. Each track of the first device accessory has a first track portion extending along the respective first sidewall portion and a second track portion extending along the respective second sidewall portion.

In some examples, when the first device accessory is in the storage position, the first and second track portions of each track are aligned for engaging corresponding ones of the third and fourth accessory attachment members of the accessory mount.

In some examples, when the first device accessory is in the storage position, the accessory mount overlies at least a portion of the first and second segments of the first device accessory.

In some examples, when the first device accessory is in the device standing position, the third and fourth accessory attachment members of the accessory mount are engageable with one of: i) corresponding ones of the first track portions, or ii) corresponding ones of the second track portions.

In some examples, the second track portions have an open end proximate the first segment for receiving corresponding ones of the third and fourth accessory attachment members. The second track portions further have a closed end opposite the open end for impeding disengagement of corresponding ones of the third and fourth accessory attachment members.

In some examples, the accessory kit further includes a second device accessory slidably engageable with the accessory mount via the first and second accessory attachment members.

In some examples, the second device accessory includes a pair of opposed mounting members spaced longitudinally apart by the first distance. Each mounting member of the second device accessory is configured to slidably engage a corresponding one of the first and second accessory attachment members of the accessory mount.

In some examples, the second device accessory includes a pair of longitudinally spaced apart second accessory sidewalls. Each mounting member of the second device accessory is provided as a track extending along a respective one of the second accessory sidewalls for receiving one of the first and second accessory attachment members of the accessory mount.

In some examples, the second device accessory includes a first segment and a second segment pivotably connected to a longitudinal edge of the first segment. The second device accessory is movable between a storage position, in which the first and second segments of the second device accessory extend generally parallel to one another, and at least a device standing position. In the device standing position, the first segment of the second device accessory is inclined relative to the second segment of the second device accessory by a stand angle.

In some examples, the stand angle is between 40 and 100 degrees.

In some examples, each second accessory sidewall has a first sidewall portion provided on the first segment and a second sidewall portion provided on the second segment. Each track of the second device accessory has a first track portion extending along the respective first sidewall portion and a second track portion extending along the respective second sidewall portion.

In some examples, when the second device accessory is in the storage position, the first and second track portions of each track are aligned for engaging corresponding ones of the first and second accessory attachment members of the accessory mount.

In some examples, when the second device accessory is in the storage position, the accessory mount overlies at least a portion of the first and second segments of the second device accessory.

In some examples, when the second device accessory is in the device standing position, the first and second accessory attachment members of the accessory attachment apparatus are engageable with one of: i) the first track portions of corresponding ones of the tracks, or ii) the second track portions of corresponding ones of the tracks.

In some examples, the second track portions have an open end proximate the first segment for receiving corresponding ones of the first and second accessory attachment members. The second track portions further include a closed end opposite the open end for impeding disengagement of corresponding ones of the first and second accessory attachment members.

In another aspect, an accessory assembly for a portable electronic device is disclosed. The accessory assembly includes an accessory mount, a device accessory slidably engageable with the accessory mount, and a user item holder releasably securable to the device accessory. The accessory mount includes a mount body and at least one device fastener affixed to the mount body for releasably securing the accessory mount to the portable electronic device. The accessory mount further includes a pair of opposed accessory attachment members including a first accessory attachment member extending from and about a first side of the mount body and a second accessory attachment member extending from and about a second side of the mount body opposite the first side of the mount body. The device accessory includes a pair of opposed mounting members. Each mounting member is configured to slidably engage a corresponding one of the first and second accessory attachment members of the accessory mount. The device accessory further includes a first segment and a second segment pivotably connected to an edge of the first segment. The device accessory is movable between a storage position, in which the first and second segments extend generally parallel to one another, and at least a device standing position, in which the first segment is inclined relative to the second segment by a stand angle. The user item holder is releasably securable to the second segment of the device accessory. The user item holder includes a pair of opposed clips for engaging opposed edges of the second segment to secure the user item holder to the second segment.

In some examples, the user item holder includes a holder body having opposed first and second holder body edges. The pair of opposed clips includes a first clip extending generally perpendicularly from the first holder body edge and a second clip extending generally perpendicular from the second holder body edge.

In some examples, the second segment has a length between the opposed edges engageable with corresponding ones of the first and second clips. The first and second clips are spaced apart by a clip gap sized to accommodate the length of the second segment.

In some examples, the holder body is resiliently flexible between planar and flexed positions, biased to the planar position, and urged to the flexed position when the second segment is received between the first and second clips.

In some examples, each of the first and second clips includes a finger that extends generally perpendicularly from a respective one of the first and second body holder edges to a finger distal end. Each finger distal end has an inwardly facing catch for engaging an underside surface of the second segment.

In some examples, each of the first and second clips includes a finger that is resiliently flexible between inward and outward positions, biased to the inward position, and urged toward the outward position when the second segment is received between the first and second clips.

In some examples, the holder body has an outer surface, and the accessory assembly further includes a storage compartment affixed to the outer surface of the holder body and configured to hold a user item.

In some examples, the storage compartment and the holder body are of unitary construction.

In some examples, the accessory assembly further includes a foam insert disposed within the storage compartment. The insert has a molded cutout that approximates the shape of the user item.

In some examples, the holder body has an inner surface opposite the outer surface. When the user item holder is secured to the second segment, the inner surface of the holder body overlies at least a portion of an outer surface of the second segment.

In some examples, the holder body has opposed third and fourth holder body edges that extend generally perpendicular to the first and second holder body edges. The third and fourth holder body edges are spaced apart by a holder body depth. The outer surface of the second segment includes a first retaining ridge and a second retaining ridge spaced apart from and extending parallel to the first retaining ridge. The first and second retaining ridges are spaced apart by a ridge gap sized to accommodate the holder body depth and prevent sliding of the user item holder when secured to the second segment.

In some examples, the outer surface of the second segment has a recessed portion between the first and second retaining ridges. When the user item holder is secured to the second segment, the holder body is seated in the recessed portion.

In some examples, the device accessory includes a pair of opposed accessory sidewalls. Each mounting member of the device accessory is provided as a track extending along a respective one of the accessory sidewalls for engaging one of the first and second accessory attachment members of the accessory mount.

In some examples, each accessory sidewall has a first sidewall portion provided on the first segment and a second sidewall portion provided on the second segment. Each track of the device accessory has a first track portion extending along the respective first sidewall portion and a second track portion extending along the respective second sidewall portion.

In some examples, the first segment includes a first segment body having a pair of opposed edges. The first sidewall portions project generally upwardly from respective ones of the opposed edges of the first segment body to define upper sidewall sections. The first sidewall portions project generally downwardly from respective ones of the opposed edges of the first segment body to define lower sidewall sections. The upper sidewall section of each first sidewall portion includes the first track portion of the respective track. The lower section of each first sidewall portion includes a further track for engaging a corresponding one of the first and second accessory attachment members of the accessory mount.

In some examples, when the device accessory is in the storage position, the first and second track portions of each track are aligned for engaging corresponding ones of the first and second accessory attachment members of the accessory mount.

In some examples, when the device accessory is in the device standing position, the first and second accessory attachment members of the accessory mount are engageable with one of: i) corresponding ones of the first track portions, ii) corresponding ones of the second track portions, or iii) corresponding ones of the further tracks.

In some examples, the first segment includes at least one device accessibility aperture. When the accessory mount is secured to the portable electronic device, the device accessory is secured to the accessory mount and the device accessory is in the storage position, the device accessibility aperture overlies one or more components of the portable electronic device to permit unimpeded functioning of those one of more components.

In some examples, the second segment includes at least one device accessibility aperture. When the accessory mount is secured to the portable electronic device, the device accessory is secured to the accessory mount and the device accessory is in the storage position, the device accessibility aperture overlies one or more components of the portable electronic device to permit unimpeded functioning of those one of more components.

### BRIEF DESCRIPTION OF DRAWINGS

For a better understanding of the described examples and to show more clearly how they may be carried into effect, reference will now be made, by way of example, to the accompanying drawings in which:

FIG. 1 is a front perspective view of an accessory assembly for a portable electronic device according to aspects of the teaching disclosed herein;

FIG. 2 is a front perspective view of an accessory mount of the accessory assembly of FIG. 1;

FIG. 3 is a front view of the accessory mount of FIG. 2;

FIG. 4 is a rear view of the accessory mount of FIG. 2 shown secured to a portable electronic device;

FIGS. 5 and 6 are perspective views of the accessory assembly of FIG. 1 shown supporting a portable electronic device in different handsfree portrait viewing positions;

FIG. 7A is a front perspective view of a device accessory of the accessory assembly of FIG. 1;

FIG. 7B is a front perspective view of a first segment of the device accessory of FIG. 7A;

FIG. 8 is a partially exploded rear perspective view of the accessory assembly of FIG. 1 including a removable item holder for holding an epinephrine injector;

FIG. 9 is a rear perspective view of the device accessory of FIG. 7A including a removable user item holder for holding a portable speaker or voice assistant device;

FIG. 10 is a rear perspective view of the accessory assembly of FIG. 1 including a removable recess cover;

FIG. 11 is a perspective view of the accessory assembly of FIG. 1 shown supporting a portable electronic device in a handsfree portrait viewing position and holding a portable speaker in front of the device screen;

FIG. 12 is a front perspective view of another device accessory that may be used in an accessory assembly according to aspects of the teaching disclosed herein;

FIG. 13 is a perspective view of an accessory assembly including the device accessory of FIG. 12 shown supporting a portable electronic device in a handsfree landscape viewing position;

FIG. 14 is a perspective view of another device accessory that may be used in an accessory assembly according to aspects of the teaching disclosed herein;

FIG. 15 is a perspective view of an accessory assembly including the device accessory of FIG. 14 shown in a filming position and supporting an external microphone and light; and

FIG. 16 is a schematic illustration of an example accessory kit, with the kit including an accessory mount and two device accessories.

### DETAILED DESCRIPTION

Various apparatuses or processes will be described below to provide an example of each claimed invention. No example described below limits any claimed invention and any claimed invention may cover processes or apparatuses that differ from those described below. The claimed inventions are not limited to apparatuses or processes having all of the features of any one apparatus or process described below or to features common to multiple or all of the apparatuses described below. It is possible that an apparatus or process described below is not an example of any claimed invention. Any invention disclosed in an apparatus or process described below that is not claimed in this document may be the subject matter of another protective instrument, for example, a continuing patent application, and the applicants, inventors, or owners do not intend to abandon, disclaim, or dedicate to the public any such invention by its disclosure in this document.

Portable electronic devices, such as smart phones and tablets, have become an integral part of society. The uses of portable electronic devices have grown rapidly over the past decade and promises to continue to do so. For example, portable electronic devices may be used to stream videos, send emails, pay bills, host video calls, take pictures, film videos, play games, message friends, etc. Use of such portable electronic devices has become so widespread that many owners keep their device within arm's reach at all times.

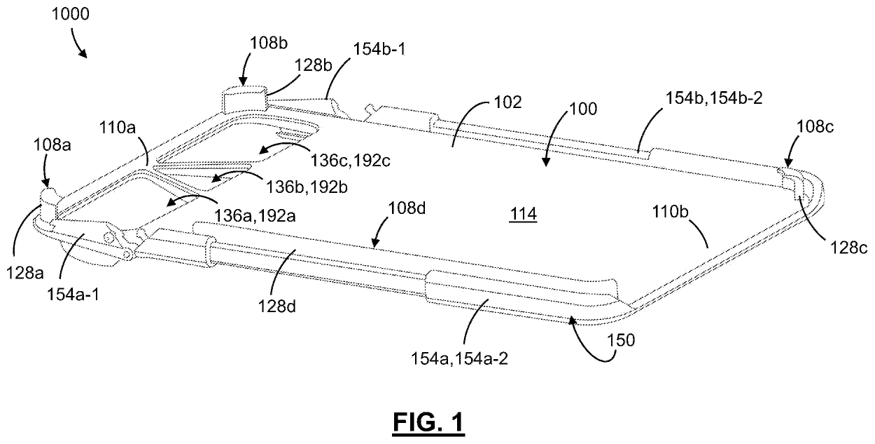

Referring to FIG. 1, an accessory assembly 1000 for a portable electronic device in accordance with aspects of the teaching disclosed herein includes features that enhance functionality and/or user enjoyment of the portable electronic device. The accessory assembly 1000 includes an accessory mount 100 and a device accessory 150 slidably engageable with the accessory mount 100. In use, the accessory mount 100 is attached to a portable electronic device (e.g., smart phone, tablet computer, etc.) and includes mounting features that allow quick connection and disconnection to the device accessory 150. The device accessory 150 is slidable relative to the accessory mount 100 and selectively movable between one or more “in-use” positions. As will be described in more detail subsequently herein, the accessory assembly 1000 may provide, among other functions, a stand for the portable electronic device, an improved grip for the portable electronic device, a shield for the screen or camera of the portable electronic device, improved call quality, and/or one or more user storage compartments.

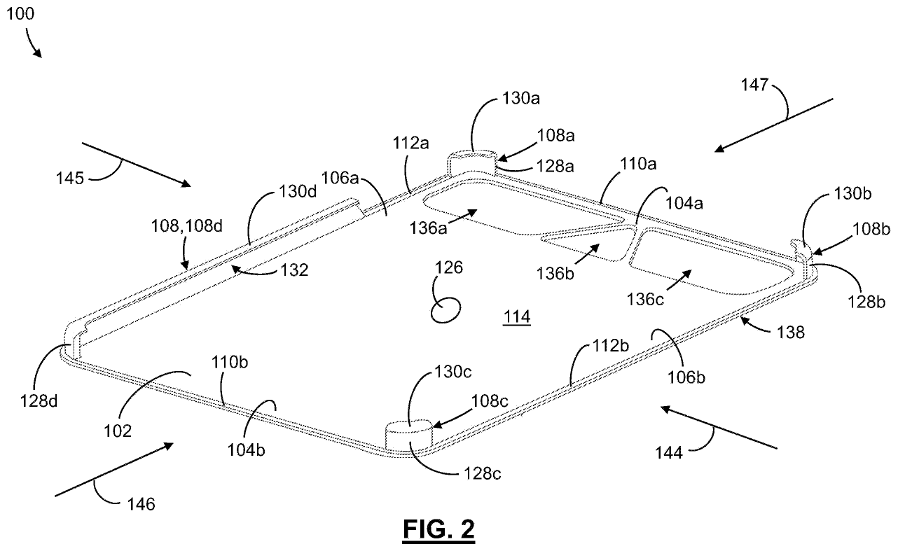

Referring to FIGS. 2-4, the accessory mount 100 includes a mount body 102 having longitudinally opposed first and second sides 104a, 104b and laterally opposed third and fourth sides 106a, 106b. The accessory mount 100 further includes a device fastener 108 affixed to the mount body 102 for releasably securing the accessory mount 100 to a portable electronic device.

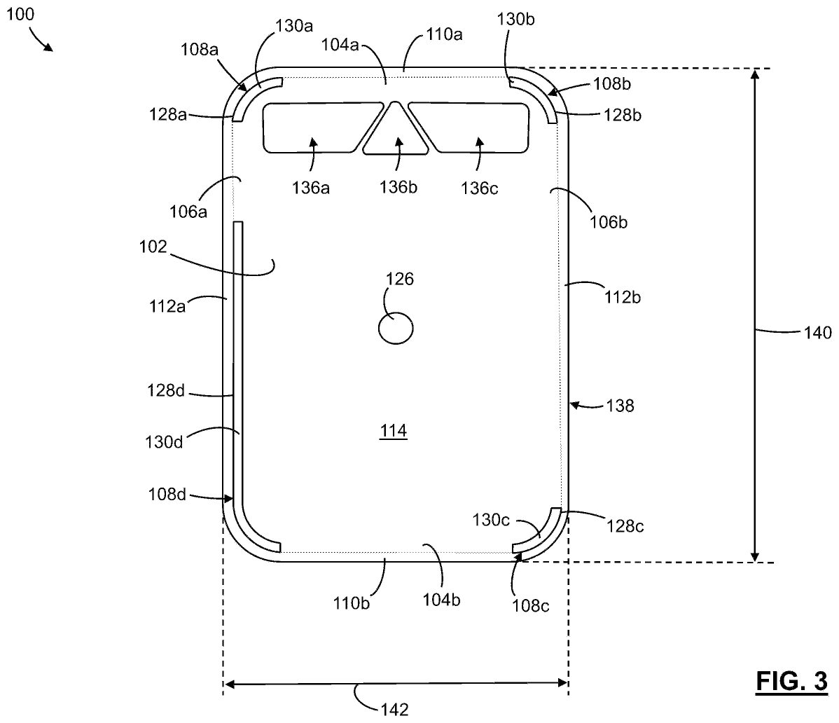

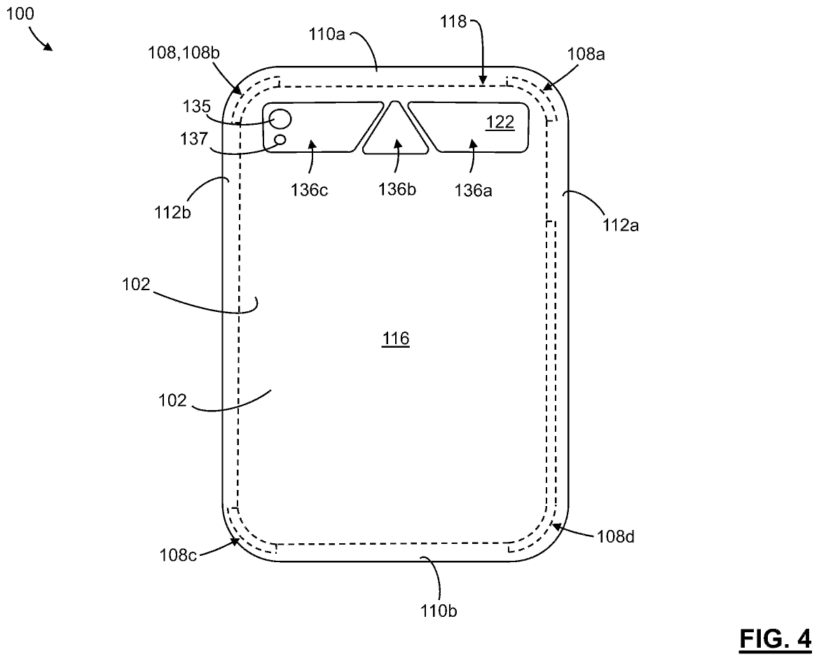

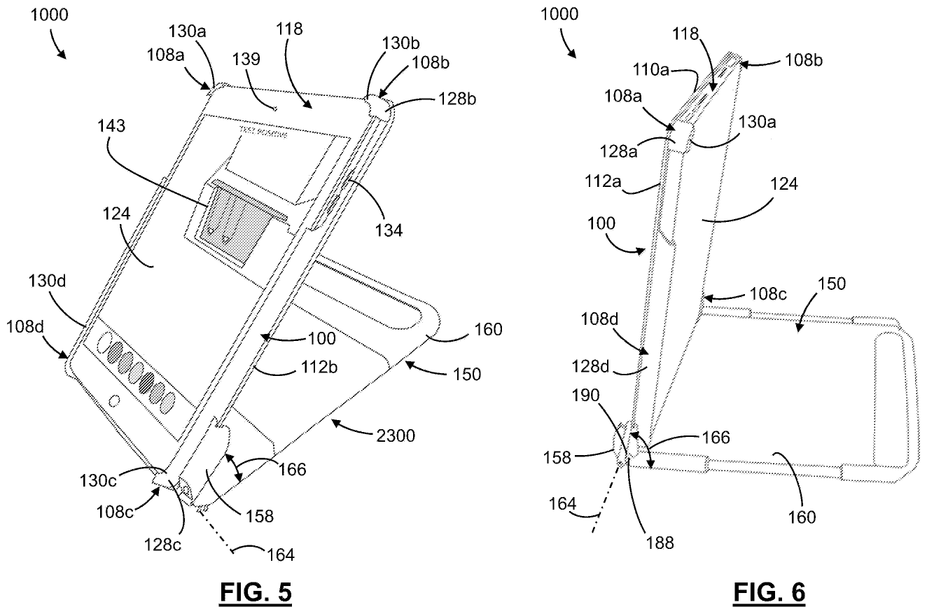

With reference to FIGS. 3 and 4, the mount body 102 includes a front surface 114 and an opposed rear surface 116. When the accessory mount 100 is secured to a portable electronic device 118, the front surface 114 of the mount body 102 at least partially overlies a rear surface 122 of the portable electronic device 118. The rear surface 122 of the portable electronic device 118 is opposite a front surface of the portable electronic device, which includes a device screen 124 (FIGS. 5 and 6).

The device fastener 108 can be any type of fastener that provides a firm connection between the accessory mount 100 and the portable electronic device 118. In some examples, the device fastener includes an adhesive for securing the accessory mount 100 to the rear surface 122 of the portable electronic device 118. Alternatively, or in addition, the device fastener may include one half of a hook and loop fastener, with the other half affixed to the rear surface 122 of the portable electronic device 118.

In the illustrated example, the device fastener 108 is in the form a clip that extends partially around a perimeter of the mount body 102. The clip includes a sidewall 128 extending generally perpendicularly from the perimeter of the mount body 102. The clip, in the illustrated example, further includes an optional device retaining ledge 130 that extends inboard from an upper edge of the sidewall 128. Referring to FIG. 2, in the illustrated example, a peripheral portion of the mount body 102, the sidewall 128 and the retaining ledge 130 define a recess 132 for receiving a peripheral portion of the portable electronic device 118 (e.g., see FIGS. 5 and 6). The sidewall 128 and the retaining ledge 130 cooperate to hold the peripheral portion of the portable electronic device 118 within the recess 132, and thereby secure the accessory mount 100 to the portable electronic device 118. In some examples, the mount body 102 is made of a resiliently flexible material to help position the peripheral portion of the portable electronic device 118 in the recess 132.

In some examples, the device fastener 108 includes a plurality of device fastener segments with each fastener segment spaced apart from an adjacent fastener segment along the perimeter of the mount body 102. Referring again to FIG. 2, in the illustrated example, the clip includes four spaced apart clip segments 108a, 108b, 108c and 108d. The clip segments 108a, 108b, 108c and 108d cooperate to releasably secure the accessory mount 100 to a portable electronic device. Each clip segment 108a, 108b, 108c and 108d includes a respective sidewall segment 128a, 128b, 128c and 128d and a respective retaining ledge portion 130a, 130b, 130c, and 130d. In the illustrated example, the clip segments 108a, 108b, 108c and 108d are positioned at respective corners of the mount body 102 to provide a secure connection with a portable electronic device.

When the accessory mount 100 is secured to a portable electronic device, space between adjacent clip segments may provide access gaps for one or more components of the portable electronic device (e.g., buttons, speakers, ports, etc.). For example, with reference to FIG. 5, space between clip segment 108b and 108c provides an access gap for a user to interact with input buttons 134 of the portable electronic device 118. In an alternative example, the clip may extend completely around the perimeter of the mount body 102. A clip that extends completely around the perimeter of the mount body 102 may provide for a stronger connection between the accessory mount 100 and a portable electronic device compared to a clip that includes a plurality of spaced apart clip segments.

Referring to FIGS. 2 and 3, in the example illustrated, the device fastener 108 further includes an optional mounting magnet 126 affixed to the mount body 102. When the accessory mount 100 is secured to a portable electronic device, the mounting magnet 126 overlies and interacts with a device magnet (not shown) of a portable electronic device to strengthen the connection between the accessory mount 100 and the portable electronic device. The magnetic attraction between the mounting magnet 126 and the device magnet can supplement the connection between the clip and the peripheral portion of the portable electronic device (e.g., making it harder to unintentionally remove the accessory mount 100 from the electronic device).

In some examples, the device fastener 108 includes one or more mounting magnets 126 and does not include the clip. The magnetic attraction between the mounting magnet 126 and the device magnet can be sufficient to firmly secure the mounting accessory 100 to the portable electronic device. The use of magnets can facilitate a swift and simple means to releasably secure the accessory mount 100 to a rear surface of a portable electronic device.

In the illustrated example, the mount body 102 optionally includes one or more device accessibility apertures 136. When the accessory mount 100 is secured to a portable electronic device, the device accessibility aperture(s) 136 overlie one or more components located on a rear surface of the portable electronic device to permit unimpeded function of those one or more components. For example, components that may be located on the rear surface of the portable electronic device include cameras, magnets, speakers, and lights. In the illustrated example, the mount body 102 includes three device accessibility apertures 136a, 136b and 136c positioned proximate the first side 104a of the mount body 102. For example, with reference to FIG. 4, the device accessibility aperture 136a overlies a rear camera 135 and flashlight 137 of the portable electronic device 118 so that the rear device camera 135 and flashlight 137 are not blocked by the mount body 102.

Referring again to FIGS. 2-4, the accessory mount 100, in the illustrated example, includes a pair of longitudinally opposed first and second accessory attachment members 110a, 110b and a pair of laterally opposed third and fourth accessory attachment members 112a, 112b. In use, the first and second accessory attachment members 110a, 110b or the third and fourth accessory attachment members 112a, 112b are slidably engaged with corresponding mounting members of a device accessory. In the illustrated example, the first accessory attachment member 110a is in the form of a lip that extends from and about the first side 104a of the mount body 102, the second accessory attachment member 110b is in the form of a lip that extends from and about the second side 104b of the mount body 102, the third accessory attachment member 112a is in the form of a lip that extends from and about the third side 106a of the mount body 102 and the fourth accessory attachment member 112b is in the form of a lip that extends from and about the fourth side 106b of the mount body 102. In an alternative example, the accessory mount 100 has only first and second accessory attachment members 110a, 110b. In another alternative example, the accessory mount 100 has only third and fourth accessory attachment members 112a, 112b.

Each of the first, second, third, and fourth accessory attachment members 110a, 110b, 112a and 112b extend along a portion of a respective side of the mount body 102. In the example illustrated, each of the first and second attachment members 110a, 110b extends respectively along the full length of the first and second sides 104a, 104b of the mount body 102. Similarly, in the illustrated example, each of the third and fourth accessory attachment members 112a, 112b extends respectively along the full length of the third and fourth sides 106a, 106b of the mount body 102. Accessory attachment members that extend along the full length of respective sides of the mount body 102 can provide a stronger engagement to a device accessory than accessory attachment members that extend along only a portion of the length. On the other hand, accessory attachment members that extend along a smaller portion of respective sides of the mount body 102 require less material than accessory attachment members that extend the full length of the respective side.

The accessory mount 102, in the illustrated example, includes an uninterrupted peripheral attachment lip 138 extending from and about the perimeter of the mount body 102. The peripheral attachment lip 138 includes the first, second, third and fourth accessory attachment members 110a, 110b, 112a and 112b. The peripheral attachment lip 138 may offer increased strength and/or durability over an example in which the accessory attachment members 110a, 110b, 112a, 112b are not interconnected. For example, the peripheral attachment lip 138 may eliminate ends of discrete accessory attachment members that may be easily snapped, bent, chipped, or otherwise damaged. The peripheral attachment lip 138, in the illustrated example, is optionally rounded at the corners of accessory mount 110 to enhance ergonomics.

An accessory mount including a pair of longitudinally opposed accessory attachment members 110 and a pair of laterally opposed accessory attachment members 112 offers enhanced versatility over an accessory mount with only one pair of accessory attachment members. In the illustrated example, a user can slidably connect the accessory mount 100 to a device accessory using either i) the first and second accessory attachment members 110a, 110b or ii) the third and fourth accessory attachment members 112a, 112b.

Referring to FIG. 2, in the illustrated example, the first and second accessory mounting members 110a, 110b of the accessory mount 100 are slidably engageable with a device accessory in either one of mounting directions 144, 145 and the third and fourth accessory mounting members 112a, 112b of the accessory mount 100 are slidably engageable with a device accessory in either one of mounting directions 146, 147. The ability of the accessory mount 100 to connect to device accessories in multiple directions, using either the first and second accessory attachment members 110a, 110b or the third and fourth accessory attachment members 112a, 112b, makes it versatile. The versatility of the accessory mount 100, in turn, opens up more uses for the connected device accessory.

Referring again to FIG. 1, when coupled to a portable electronic device via the accessory mount 100, the device accessory 150 can enhance use of the portable electronic device. Some device accessories include stand features that can be deployed to support the portable electronic device in a handsfree viewing position. Alternatively, or in addition, some device accessories include sun shield features that can be deployed to limit glare on the device screen. Alternatively, or in addition, some device accessories include a privacy shield that can be deployed to impede those nearby from viewing the device screen. Alternatively, or in addition, some device accessories include storage features for holding user items.

Referring to FIG. 3, in the illustrated example, the first and second accessory attachment members 110a, 110b are spaced longitudinally apart by a first distance 140. In the illustrated example, the third and fourth accessory attachment members 112a, 112b are spaced laterally apart by a second distance 142. In the illustrated example, the second distance 142 is shorter than the first distance 140. In other examples, the first and second distances 140, 142 may be generally equal.

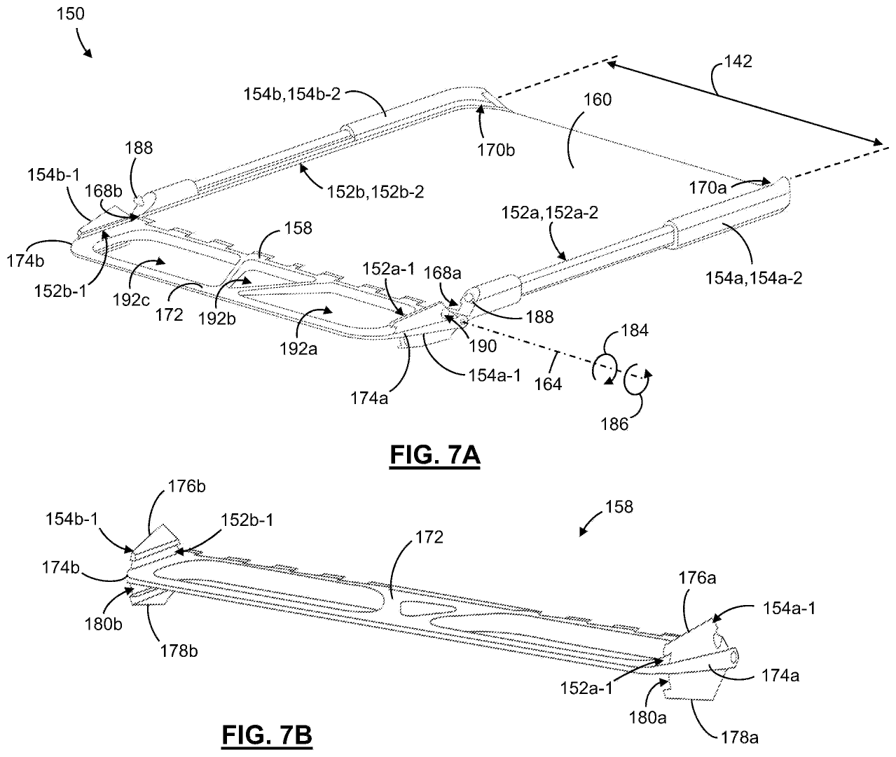

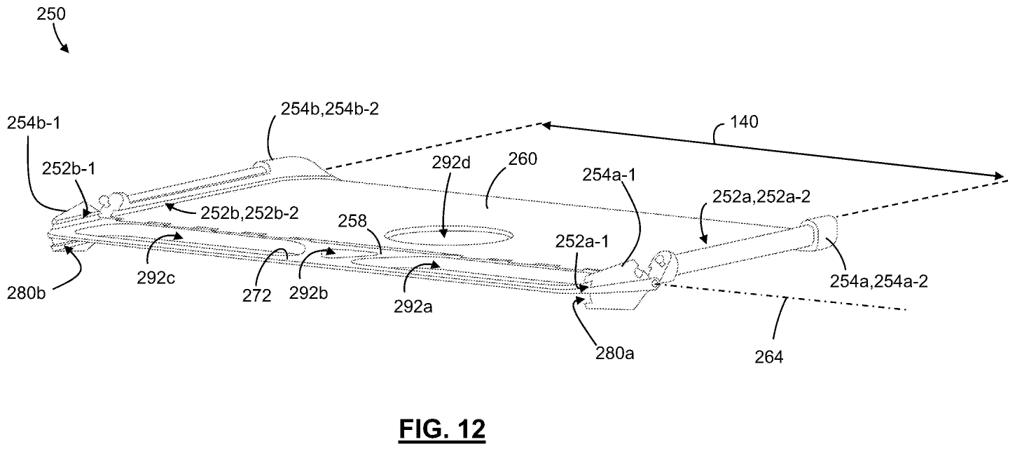

The accessory mount 100, in the illustrated example, can slidably engage any device accessory that includes a pair of mounting members spaced apart by the first distance 140 or the second distance 142. Referring to FIG. 7A, the device accessory 150 includes mounting members 152a, 152b that are spaced apart by the second distance 142. Referring to FIG. 12, the device accessory 250 includes the mounting members 252a, 252b that are spaced apart by the first distance 140. Hence, the accessory mount 100, in the illustrated example, can slidably engage both example device accessories 150, 250.

With two pairs of accessory attachment members 110, 112, the user is given flexibility to select and connect a device accessory that aligns with the intended use of their portable electronic device. Environmental factors may also impact which device accessory the user chooses to connect to the accessory mount 100 (e.g., the location of sun, the location of potential eavesdroppers, etc.). As an example, the device accessory 150 of FIG. 7A may be well suited for movie streaming because it can be positioned to support the electronic device in a handsfree landscape viewing position. As another example, the device accessory 250 of FIG. 12 may be well suited for outdoor video calls because it can be positioned to shield the sun from overhead when the device screen is in a portrait orientation.

As an example, the accessory mount 100 can slidably engage the device accessory 150 of FIG. 7A in either the third mounting direction 146 or the fourth mounting direction 147. The device accessory 150, in the illustrated example, includes mounting members 152a, 152b which slidably engage corresponding ones of the laterally opposed third and fourth accessory attachment members 112a, 112b. As another example, the accessory mount 100 can slidably engage the device accessory 250 of FIG. 12 in either the first mounting direction 144 or the second mounting direction 145. The device accessory 250, in the illustrated example, includes mounting members 252a, 252b which slidably engage corresponding ones of the longitudinally opposed first and second accessory attachment members 110a, 110b. When connected, each of the exemplary device accessories 150, 250 can be selectively deployed to provide stand, sun shielding, and/or screen privacy functions for the portable electronic device.

In some cases, an accessory mount 100 with two pairs of accessory attachment members 110, 112 allows a user to quickly swap accessory devices between different tasks. For example, when switching from a phone call to video streaming, the user may disconnect the device accessory 150 (FIG. 7A) from the pair of laterally opposed mounting members 112a, 112b and connect the device accessory 250 (FIG. 12) via the pair of longitudinally opposed mounting members 110a, 110b.

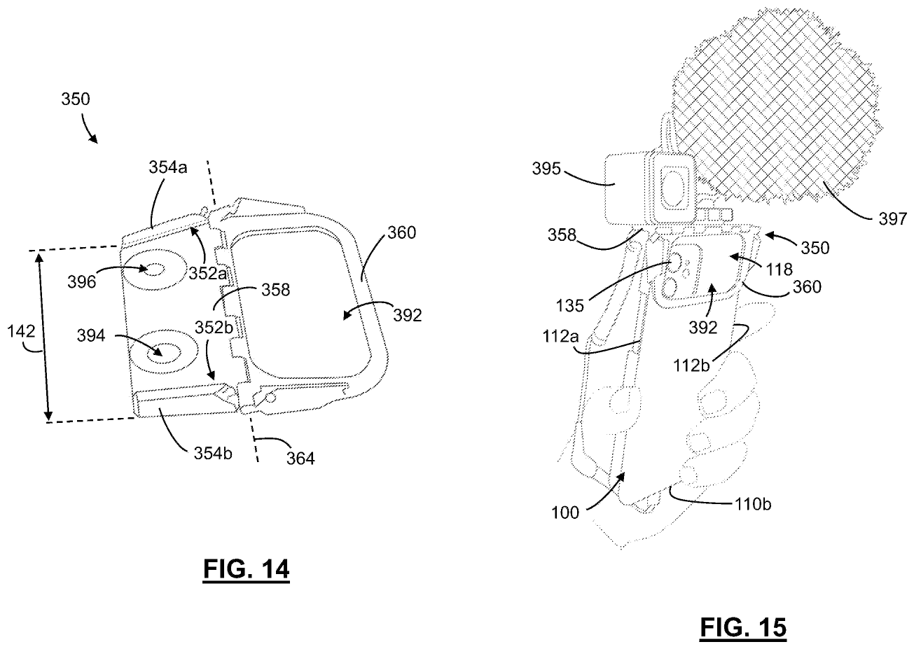

Selecting the preferred device accessory for attachment to the accessory mount 100 can be based on more than the orientation of the device screen. In some examples, device accessories are tailored for specific uses. For example, FIGS. 14 and 15 illustrate an example device accessory 350 customized for filming videos. The device accessory 350 includes a light mount aperture 394 and a microphone mount aperture 396. The light mount aperture 394 holds an external light 395 and the microphone mount aperture 396 holds an external microphone 397 while the portable electronic device is used to film a video. The device accessory 350, in the illustrated example includes spaced apart mounting members 352a, 352b configured to slidably engage corresponding ones of the laterally opposed third and fourth accessory attachment members 112a, 112b of the accessory mount 100. For example, when switching from editing to filming, a user may disconnect the device accessory 150 from the pair of laterally opposed mounting members 112a, 112b and connect the device accessory 350 via the pair of laterally opposed mounting members 112a, 112b.

Referring again to FIG. 3, in the illustrated example, each of the first, second, third and fourth accessory attachment members 110a, 110b, 112a, and 112b extends outboard of the sidewall 128 of the device fastener 108. In some examples, each accessory attachment member 110a, 110b, 112a and 112b extends between 1 mm and 5 mm outboard of the sidewall 128. Accessory attachment members 110a, 110b, 112a and 112b that extend between 1 mm and 5 mm outboard of the sidewall 128 provide a firm and stable connection with a device accessory and do not overly enlarge the footprint of the portable electronic device in a manner detrimental to user convenience. In some examples, the accessory attachment members 110a, 110b, 112a and 112b may additionally, or alternatively, extend rearwardly from respective sides of the mount body 102.

In some examples, the mount body 102, the device fastener 108, and the accessory attachment members 110, 112 are of integral, unitary construction. For example, the accessory mount 100 may be formed as a single unit with known injection molding, thermoforming, or 3D printing techniques. In other examples, the mount body 102, the device fastener(s) 108 and the attachment members 110, 112 are formed separately and then assembled into the accessory mount 100.

In the illustrated example, front and rear surfaces of the accessory attachment members 110a, 110b, 112a, 112b are flush with the respective front and rear surfaces 114, 116 of the mount body 102. This may simplify manufacturing the accessory mount 100. In some examples, front and rear surfaces of the accessory attachment members 110a, 110b, 112a, 112b are not flush with the respective front and rear surfaces 114, 116 of the mount body 102.

Referring to FIG. 7A, the device accessory 150 includes a pair of opposed mounting members 152a, 152b configured to slidably engage corresponding ones of the third and fourth accessory attachment members 112a, 112b of the accessory mount 100 (FIG. 2). In the illustrated example, the mounting members 152a and 152b are spaced apart by distance 142. With reference to FIGS. 3 and 7A, the distance 142 corresponds to the distance separating the pair of laterally opposed first and second accessory attachment members 112a, 112b of the accessory mount 100. This allows one of the mounting members 152a, 152b to slidably engage one of the third and fourth accessory attachment members 112a, 112b while the other of the mounting members 152a, 152b slidably engages the other of the third and fourth accessory attachment members 112a, 112b.

Referring again to FIG. 7A, in the illustrated example, the device accessory 150 includes a pair of laterally spaced apart accessory sidewalls 154a, 154b. In the illustrated example, each mounting member 152a, 152b is in the form of a track that extends along a respective one of the accessory sidewalls 154a, 154b for receiving one of the third and fourth accessory attachment members 112a, 112b of the accessory mount 100 (FIG. 3).

The device accessory 150, in the illustrated example, further includes a first segment 158 and a second segment 160 pivotably connected to an edge of the first segment 158. The accessory sidewalls 154a, 154b, in the illustrated example, have respective first sidewall portions 154a-1, 154b-1 provided on the first segment 158 and respective second sidewall portion 154a-2, 154b-2 provided on the second segment 160. In the illustrated example, each track 152a, 152b includes a first track portion 152a-1, 152b-1 extending along a respective one of the first sidewall portions 154a-1, 154b-1 and a second track portion 152a-2, 152b-2 extending along a respective one of the second sidewall portion 154a-2, 154b-2.

The device accessory 150 is movable between a storage position (FIG. 7A) and at least a device standing position (e.g., FIGS. 5 and 6). In the illustrated example, the device accessory 150 is movable between the storage position and a device standing position by pivoting one of the first and second segments 158, 160 about a pivot axis 164 relative to the other of the first and second segments 158, 160. When the device accessory 150 is in the storage position, the first and second segments 158, 160 extend generally parallel to one another (i.e., the first and second segments 158, 160 extend in the same plane).

With reference to FIGS. 3 and 7A, the first track portions 152a-1, 152b-1 are open on both ends to allow corresponding ones of the third and fourth accessory attachment members 112a, 112b to slide therethrough. In the illustrated example, the second track portions 152a-2, 152b-2 have respective open ends 168a, 168b proximate the first segment 158 for receiving corresponding ones of the third and fourth accessory attachment members 112a, 112b. The second track portions 152a-2, 152b-2, in the illustrated example, also have respective closed ends 170a, 170b opposite the open ends 168a, 168b. The closed ends 170a, 170b of respective second track portions 152a-2, 152b-2 impede disengagement of corresponding ones of the third and fourth accessory attachment members 112a, 112b (e.g., by oversliding the device accessory 150 relative to the accessory mount 100). The device accessory 150 can be disengaged from the accessory mount 100 by sliding either the device accessory 150 or the accessory mount 100 relative to the other in a direction opposite to the one used to engage the device accessory 150 and the accessory mount 100.

When the device accessory 150 is in the storage position, the first track portions 152a-1, 152b-1 and the respective second track portions 152a-2, 152b-2 are aligned for engaging corresponding ones of the third and fourth accessory attachment members 112a, 112b. The alignment of track portions in the storage position allows the third and fourth accessory attachment members 112a, 112b to respectively slide between the first track portions 152a-1, 152b-1 and the second track portions 152a-2, 152b-2.

When the device accessory 150 is in the storage position, the accessory mount 100 is at least partially stowable within the device accessory 150. For example, with reference to FIG. 1, the accessory mount 100 is fully stowed within the device accessory 150. Such a stowed arrangement can be space saving when the device accessory 150 is not being used.

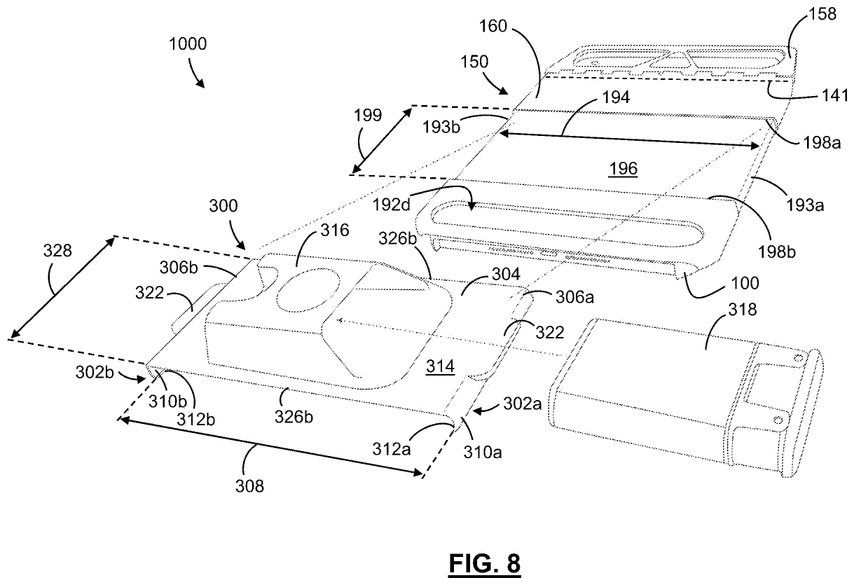

When the device accessory 150 is in a device standing position, the first segment 158 is inclined relative to the second segment 160 by a stand angle. In some example, the stand angle may be set by one or more optional rear hinge stops 141 (see FIG. 8) which consist of one or more narrow protrusions proximate to the hinge that limit further pivoting. In some examples, the stand angle is between 40 and 100 degrees. More preferably, the stand angle is between 50 and 90 degrees. Stand angles within these ranges provide for ergonomic viewing angles of the device screen. In the illustrated examples of FIGS. 5 and 6, the stand angle 166 is about 50 degrees and about 90 degrees, respectively. The device accessory 150 supports the portable electronic device 118 in a handsfree portrait viewing position when in a device standing position. When in the portrait viewing position, the user may also use the front-facing camera 139 (see FIG. 5) for video calls, such as for telemedicine or telehealth sessions.

The handsfree portrait viewing position illustrated in FIG. 5 also facilitates the use of the rear device camera 135 (FIG. 4). Referring to FIG. 5, an application executed by the processor of the electronic device 118 is using an image 143 taken by the rear device camera 135 to analyze a medical sample. In the illustrated example, the application has detected a positive result florescent glow of a CRISPR test for detecting COVID-19. Other non-medical rear camera stand uses are contemplated, such as, for example, barcode or QR scanning at front security desks.

Referring to FIG. 7A, in the illustrated example, the first segment 158 is pivotable relative to the second segment 160 in both a clockwise direction 184 and a counterclockwise direction 186 about the pivot axis 164. In some examples, the stand angle is predetermined by one or more pivot stops that inhibit further pivoting in either the clockwise or counterclockwise directions 184, 186. In the illustrated example, pivoting in the counterclockwise direction 186 is inhibited when portions of the first and second segments 158, 160 proximate the pivot axis 164 (e.g., see FIG. 5). In the illustrated example, pivoting in the clockwise direction 184 is inhibited when tabs 188 positioned at ends of the respective second sidewall portions 154a-2, 154b-2 snap into corresponding sockets 190 positioned at ends of the respective first sidewall portions 154a-1, 154b-1 (e.g., see FIG. 6).

When the device accessory 150 is in a device standing position, the third and fourth accessory attachment members 112a, 112b of the accessory mount 100 are engageable with one of i) corresponding ones of the first track portions 152a-1, 152b-1 or ii) corresponding ones of the second track portions 152a-2, 152b-2. For example, with reference to FIGS. 5 and 6, the third and fourth attachment members 112a, 112b are engaged with corresponding ones of the first track portions 152a-1, 152b-1.

Although FIGS. 5 and 6 depict the device accessory 150 in exemplary device standing positions, the device accessory 150 is just as easily movable to a device screen privacy position, in which one of the first and second segments 158, 160 is positioned to impede those nearby from viewing the device screen. Further, the device accessory 150 may be movable to a sun shielding position, in which one of the first and second segments 158, 160 is positioned to limit glare on the device screen. Further still, the device accessory 150 may be movable to a mouth concealing position, in which one of the first and second segments 158, 160 is positioned to conceal the user's mouth during a call.

In some examples, the first segment 158 and/or the second segment 160 of the device accessory 150 include one or more optional device accessibility apertures. The device accessibility apertures of the device accessory 150 are positioned so that they at least partially overlie corresponding device accessibility apertures of the accessory mount 100 when the device accessory 150 is in the storage position and stowed within the accessory mount 100. This alignment may permit components of the device (e.g., cameras, lights, magnets, etc.) to function without impediment.

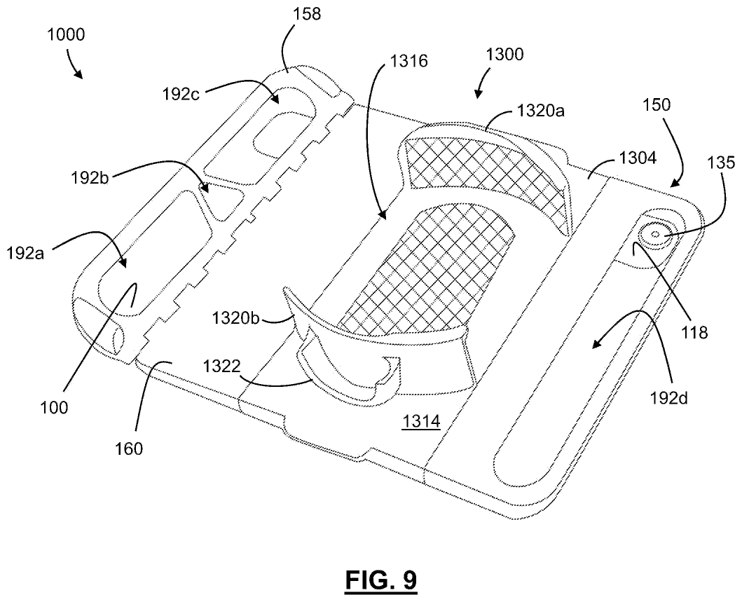

Referring to FIG. 7A, in the illustrated example, the first segment 158 includes three device accessibility apertures 192a, 192b, and 192c. Each device accessibility aperture 192a, 192b, 192c is positioned to align with a corresponding one of the device accessibility apertures 136a, 136b, and 136c of the accessory mount 100 when the device accessory 150 is in the storage position and stowed within the accessory mount 100 (e.g., see FIG. 1). In some examples, as exemplified in FIG. 9, the second segment 160 includes an optional device accessibility aperture 192d to allow for unimpeded use of the rear device camera 135.

Referring to FIG. 8, the accessory assembly 1000 may optionally include a user item holder 300 that is releasably securable to the device accessory 150. The user item holder 300 allows one or more user items to conveniently be held with the portable electronic device. The user item holder 300 includes opposed first and second clips 302a, 302b. In the illustrated example, the first and second clips 302a, 302b engage corresponding ones of opposed edges 193a, 193b of the second segment 160 to secure the user item holder 300 to the device accessory 150. The user item holder 300 can be removed from the accessory assembly 1000 when it is not needed (e.g., see FIGS. 5 and 6).

In the illustrated example, the user item holder 300 includes a holder body 304 having opposed first and second holder body edges 306a, 306b. In the illustrated example, the first clip 302a extends generally perpendicularly from the first holder body edge 306a and a second clip 302b extends generally perpendicularly from the second holder body edge 306b. When the user item holder 300 is secured to the second segment 160, an inner surface of the holder body 304 overlies at least a portion of an outer surface of the second segment 160.

In the illustrated example, the second segment 160 has a length 194 between the opposed edges 193a, 193b. The first and second clips 302a, 302b are spaced apart by a clip gap 308 sized to accommodate the length 194 of the second segment 160. In some examples, the clip gap 308 may be the same as the length 194, or slightly shorter than the length 194, to provide a snug fit of the user item holder 300 to the second segment 160 of the device accessory 150.

In the illustrated example, the holder body 304 is resiliently flexible between planar and flexed positions. The holder body 304 is biased to the planar position and is urged to the flexed position when the second segment 160 is received between the first and second clips 302a, 302b. The holder body 304 can be urged to the flexed position to make the clip gap 308 slightly longer than the length 194 between opposed edges 193a, 193b of the second segment 160, thereby easing connection of the item holder 300 to the second segment 160.

In the illustrated example, each of the first and second clips 302a, 302b includes a respective finger 310a, 310b. The fingers 310a, 310b extend generally perpendicularly from a respective one of the first and second body holder edges 306a, 306b to a finger distal end. Each finger distal end has an inwardly facing catch 312a, 312b for engaging an underside surface of the second segment 160.

In some examples, each of the fingers 310a, 310b are resiliently flexible between inward and outward positions. The fingers 310a, 310b may be biased to the inward position and may be urged toward the outward position when the second segment 160 is received between the first and second clips 302a, 302b. The clip gap 308 is slightly enlarged as the fingers 310a, 310b are urged to the outward position, thereby giving the inwardly facing catches 312a, 312b sufficient clearance to pass corresponding ones of the opposed edges 193a, 193b.

In the illustrated example, the user item holder 300 optionally includes removal tabs 322 to facilitate release of the user item holder 300 from the device accessory 150. The tabs 322, in the illustrated example, extend outboard from respective ones of first and second holder body edges 306a, 306b to provide a convenient user grip. For example, to disconnect the user item holder 300, a user may grab one (or both) of the tabs 322 to move the resiliently flexible holder body 304 into a flexed position. When the holder body 304 is in the flexed position, the clip gap 308 is enlarged to give the inwardly facing catches 312a, 312b sufficient clearance to pass back across corresponding ones of the opposed edges 193a, 193b.

Referring still to FIG. 8, the holder body 304 has an outer surface 314. The user item holder 300, in the illustrated example, includes a storage compartment 316 affixed to the outer surface 314 of the holder body 304. In the illustrated example, the storage compartment 316 and the holder body 304 are of integral, unitary construction. In other examples, the storage compartment 316 is affixed to the outer surface 314 of the body holder 304 with a suitable mechanical fastener, e.g., hook and loop fasteners, clips, magnets, or a combination thereof.

The storage compartment 316 can be configured to hold one or more user items. Users may enjoy the convenience of holding various items with their electronic device. In some examples, the storage compartment 316 can be dimensioned to hold a specific user item, such as, for example, an external battery for charging the portable electronic device. In the illustrated example, the storage compartment 316 is dimensioned to hold an epinephrine auto-injector 318, specifically, a Kaleo™ Auvi-Q™. A user can slide the epinephrine auto-injector 318 in and out of the storage compartment as needed. Since users carry their electronic device everywhere, having the epinephrine auto-injector 318 stored on the portable electronic device makes it readily available when needed. In another example, the storage compartment 316 may be configured to hold a PillPack™ medication container. Such a storage compartment may facilitate adherence to a medication regime, for example.

In some examples, the user item 300 includes a foam insert positionable within the storage compartment 316. The foam insert may have a molded cut out that approximates the shape of a specific user item. For example, the cut out may correspond to the shape of a charging case for wireless earphones. Such a foam insert would allow the charging case to be held within the storage compartment 316 without rattling (and add protection).

In the illustrated example, the second segment 160 optionally includes a first retaining ridge 198a and a second retaining ridge 198b spaced apart from and extending parallel to the first retaining ridge 198a. The holder body 304, in the illustrated example, has opposed third and fourth holder body edges 326a, 326b that extend generally perpendicularly to the first and second holder body edges 306a, 306b. The third and fourth holder body edges 326a, 326b are spaced apart by a holder body depth 328. The first and second retaining ridges 198a, 198b of the second segment 160 are spaced apart by a ridge gap 199 sized to accommodate the holder body depth 328. When the user item holder 300 is secured between the first and second retaining ridges 198a, 198b of the second segment 160, the first and second retaining ridge 198a, 198a abut corresponding third and fourth body holder edges 326a, 326b to inhibit sliding of the user item holder 300.

In the illustrated example, the second segment 160 further includes a recessed portion 196 between the first and second retaining ridges 198a, 198b. When the user item holder 300 is seated in the recessed portion 196, the holder body 304 is flush with the non-recessed portion of the second segment 160. This may prevent the holder body 304 from catching or snagging on objects, especially when removed from the user's pocket or purse.

Referring to FIG. 9, a user item holder 1300 has some similarity to the user item holder 300, with like features identified by like reference characters, incremented by 1000. The user item holder 1300 includes a storage compartment 1316 affixed to the outer surface 1314 of the holder body 1304. In the illustrated example, the storage compartment 1316 is configured to hold a portable speaker or voice assistant device (e.g., Amazon Echo™, Google Nest™, etc.). The storage compartment 1316, in the illustrated example, is defined by curved and opposed retaining members 1320a, 1320b that extending upwardly from the outer surface 1314. The opposed retaining members 1320a, 1320b hold a portable speaker or a voice assistant device positioned therebetween in a friction fit. In the illustrated example, the retaining member 1320b includes a curved retaining slot 1322 specifically dimensioned to receive and hold a location tracker, such as a GPS tag or Apple AirTag™. Other user items which are contemplated to be held by the storage compartment 1316 include styluses, such as the Apple Pencil2™, or remote patient monitoring (RPM) equipment, such as TytoCare™ or KardiaMobile™.

Comparing FIG. 9 to FIG. 8, the portable electronic device 118 is connected to the accessory assembly 1000 in an inverted orientation. Referring to FIG. 9, the device accessibility aperture 192d of the second segment 160 overlies the rear device camera 135 to allow for its unobstructed use. FIG. 9 illustrates the first segment 158 of the device accessory 150 disengaged from the accessory mount 100 and slightly pivoted away from the electronic device 118 relative to the second segment 160. The first segment 158 of the device accessory 150 may serve as a user handle when the first segment 158 is disengaged from the accessory mount 100. For example, a user may use one or more of the apertures 192a, 192b, 192c as finger openings to improve grip. When the first segment 158 is disengaged from the accessory mount 100, any of the apertures 192a, 192b, 192c of the first segment 158 may be used to hang the portable electronic device 118 on a hook.

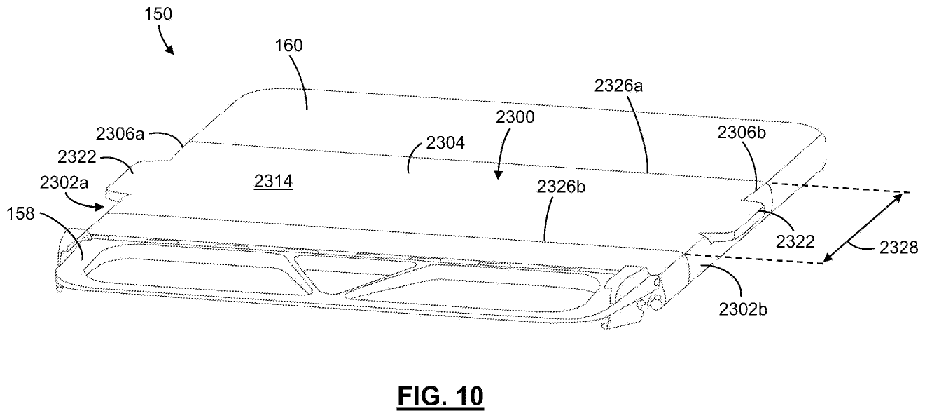

Referring to FIG. 10, a recess cover 2300 has some similarity to the user item holder 300, with like features identified by like reference characters. The recess cover 2300 includes opposed first and second clips 2302a, 2302b and optional removal tabs 2322. The recess cover 2300 includes a cover body 2304 having an outer surface 2314 and opposed first and second cover body edges 2306a, 2306b. In the illustrated example, the first clip 2302a extends generally perpendicularly from the first cover body edge 2306a and a second clip 2302b extends generally perpendicularly from the second cover body edge 2306b. The first and second clips 2302a, 2302b engage corresponding ones of opposed edges 193a, 193b of the second segment 160 to secure the recess cover 2300 to the device accessory 150.

The cover body 2304, in the illustrated example, has opposed third and fourth cover body edges 2326a, 2326b that extend generally perpendicularly to the first and second cover body edges 2306a, 2306b. The third and fourth cover body edges 2326a, 2326b are spaced apart by a cover body depth 2328 that corresponds to the ridge gap 199 (FIG. 8). The recess cover 2300 is securable to the second segment 160 to cover (i.e., fill in) the recessed portion 196 (FIG. 8). When secured, the outer surface 2314 of the cover body 2304 is flush with the non-recessed portion of the second segment 160. This may prevent the cover body 2304 from catching or snagging on objects, especially when removed from the user's pocket or purse. The recess cover 2300 can be removed when a user wishes to connect a user item holder (e.g., the user item holders 300 and 1300 of FIGS. 8 and 9, respectively).

Referring to FIG. 7B, the first segment 158 of the device accessory 150 includes a first segment body 172 having a pair of opposed edges 174a, 174b. In the illustrated example, the first sidewall portions 154a-1, 154b-1 project generally upwardly from respective ones of the opposed edges 174a, 174b of the first segment body 172 to define upper sidewall sections 176a, 176b. The first sidewall portions 154a-1, 154b-1, in the illustrated example, also project generally downwardly from respective ones of the opposed edges 174a, 174b of the first segment body 172 to define lower sidewall sections 178a, 178b.

Each upper sidewall section 176a, 176b includes the first track portion 152a-1, 152b-1 of the respective track 152a, 152b. Each lower section 178a, 178b includes a further track 180a, 180b for engaging a corresponding one of the third and fourth accessory attachment members 112a, 112b of the accessory mount 100 (FIG. 1). The further tracks 180a, 180b increase the ways in which the device accessory 150 can be attached to the accessory mount 100 (FIGS. 2 and 3) and opens up more potential uses of the accessory assembly. Referring again to FIG. 12, the first segment 258 of the device accessory 250 has a similar structure, except that further tracks 280a, 280b are engageable with corresponding ones of the first and second accessory attachment members 110a, 110b of the accessory mount 100 (FIG. 1).

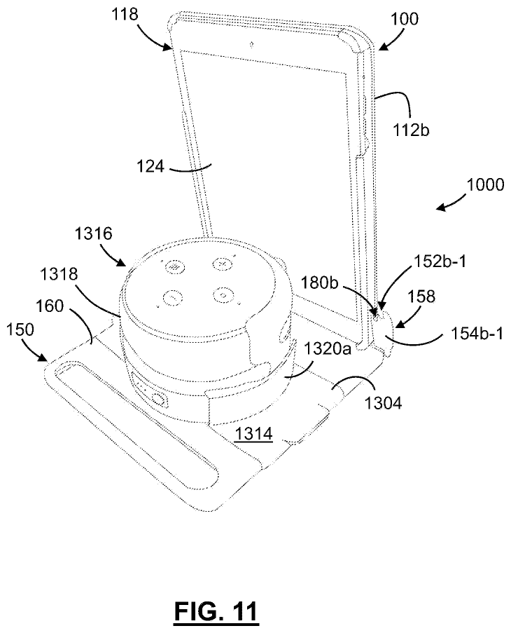

When the device accessory 150 is in a device standing position, the third and fourth accessory attachment members 112a, 112b are engageable with any one of: i) corresponding ones of the first track portions 152a-1, 152b-1, ii) corresponding ones of the second track portions 152a-2, 152b-2, or iii) corresponding ones of the further tracks 180a, 180b. For example, with reference to FIG. 11, the third and fourth accessory attachment members 112a, 112b are engaged with respective ones of the further tracks 180a, 180b (engagement between the third accessory attachment member 112a and the further track 180a is hidden by the portable speaker 1318). Engagement with the further tracks 180a, 180b allows the storage compartment 1316 of the user item holder 1300 to be positioned forward of the device screen 124, thereby permitting portable speaker 1318 held by the storage compartment 1316 to be positioned forward of the device screen 124. This arrangement can improve streaming and video watching by giving the sound emitted from the portable speaker 1318 an unobstructed path to the user's ear (i.e., the emitted sound waves are not blocked by the electronic device 118). Alternatively, the storage compartment 316 may hold a voice assistant device forward of the device screen 124, in which the case the user's voice commands will have an unobstructed path the voice assistant device.

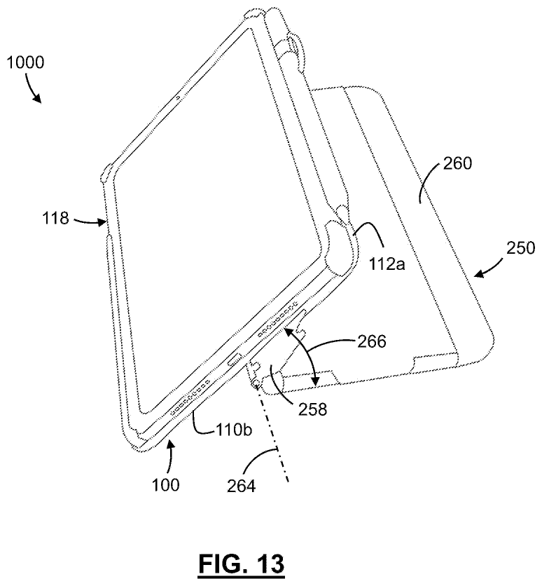

Referring to FIGS. 12 and 13, another example device accessory 250 according to aspects of the present teaching has some similarity to the device accessory 150, with like features identified by like reference characters, incremented by 100.

Referring to FIG. 12, the device accessory 250 includes a pair of opposed mounting members 252a, 252b in the form of tracks. In the illustrated example, the mounting members 252a and 252b are spaced apart by distance 140. With reference to FIGS. 3 and 12, the distance 140 corresponds to the distance separating the pair of longitudinally opposed first and second accessory attachment members 110a, 110b of the accessory mount 100. This allows one of the mounting members 252a, 252b to slidably engage one of the first and second accessory attachment members 110a, 110b while the other of the mounting members 252a, 252b slidably engages the other of the first and second accessory attachment members 110a, 110b.

Comparing FIG. 12 to FIG. 7A, the device accessory 250 differs from the device accessory 150 primarily in the arrangement of mounting members. The mounting members 252a, 252b of the device accessory 250 are longitudinally spaced apart for slidable engagement with corresponding ones of the first and second mounting members 110a, 110b of the accessory mount 100 (FIG. 3). On the other hand, the mounting members 152a, 152b of the device accessory 150 are laterally spaced apart for slidable engagement with corresponding ones of the third and fourth mounting members 112a, 112b of the accessory mount 100 (FIG. 3).

Referring again to FIG. 12, the device accessory 250 includes a pair of longitudinally spaced apart accessory sidewalls 254a, 254b. In the illustrated example, each track 252a, 252b extends along a respective one of the accessory sidewalls 254a, 254b for receiving one of the first and second accessory attachment members 110a, 110b of the accessory mount 100 (FIG. 3).

The device accessory 250, in the illustrated example, further includes a first segment 258 and a second segment 260 pivotably connected to an edge of the first segment 258. In the illustrated example, the device accessory 250 is movable between a storage position and at least a device standing position by pivoting one of the first and second segments 258, 260 about a pivot axis 264 relative to the other of the first and second segments 258, 260.

Referring to FIG. 12, when the device accessory 250 is in the storage position, the first and second segments 258, 260 extend generally parallel to one another (i.e., the first and second segments 258, 260 extend in the same plane). The accessory mount 100 (FIG. 2) can be stowed within the device accessory 250 when the device accessory 250 is in the storage position. Such a stowed arrangement can be space saving when the device accessory 250 is not being used.

Referring to FIG. 13, when the device accessory 250 is in a device standing position, the first segment 258 is inclined relative to the second segment 260 by a stand angle 266. The device accessory 250 supports the portable electronic device 118 in a handsfree landscape viewing position when in a device standing position. Like the device accessory 150, the device accessory 250 is also movable into a device screen privacy position, a sun shielding position, or a mouth concealing position by pivoting one of the first and second segments 258, 260 about the pivot axis 264 relative to the other of the first and second segments 258, 260.

Referring again to FIG. 12, the accessory sidewalls 254a, 254b, in the illustrated example, have respective first sidewall portions 254a-1, 254b-1 provided on the first segment 258 and respective second sidewall portion 254a-2, 254b-2 provided on the second segment 260. In the illustrated example, each track 252a, 252b includes a first track portion 252a-1, 252b-1 extending along a respective one of the first sidewall portions 254a-1, 254b-1 and a second track portion 252a-2, 252b-2 extending along a respective one of the second sidewall portion 254a-2, 254b-2.

In the illustrated example, the device accessory includes four device accessibility apertures 292a, 292b, 292c and 292d. The device accessibility apertures 292a, 292b and 292c are positioned on the first segment 258. The device accessibility aperture 292d is positioned on the second segment 260. When the device accessory 250 is in the storage position and stowed within the accessory mount 100, the device accessibility aperture 292d may overlie a corresponding aperture on the accessory mount 100 to allow a device logo to be seen.

Referring to FIGS. 14 and 15, another example device accessory 350 according to aspects of the present teaching has some similarity to the device accessory 150, with like features identified by like reference characters, incremented by 200. The device accessory 350 can be connected to the accessory mount 100 (FIGS. 2 and 3) to assist a user filming a video with their portable electronic device.

Referring to FIG. 14, the device accessory 350 includes a pair of opposed mounting members 352a, 352b in the form of tracks. In the illustrated example, the mounting members 352a and 352b are spaced apart by distance 142. With reference to FIGS. 3 and 14, the distance 142 corresponds to the distance separating the pair of laterally opposed third and fourth accessory attachment members 112a, 112b of the accessory mount 100. This allows one of the mounting members 352a, 352b to slidably engage one of the third and fourth accessory attachment members 112a, 112b while the other of the mounting members 352a, 352b slidably engages the other of the third and fourth accessory attachment members 112a, 112b. In alternative examples, the device accessory 350 may include mounting members that are spaced apart for slidable engagement with corresponding ones of the longitudinally opposed first and second accessory attachment members 110a, 110b.

Referring again to FIG. 14, the device accessory 350 includes a pair of longitudinally spaced apart accessory sidewalls 354a, 354b. In the illustrated example, each track 352a, 352b extends along a respective one of the accessory sidewalls 354a, 354b for receiving one of the third and fourth accessory attachment members 112a, 112b of the accessory mount 100 (FIG. 3).

The device accessory 350, in the illustrated example, further includes a first segment 358 and a second segment 360 pivotably connected to an edge of the first segment 358. In the illustrated example, the device accessory 350 is movable between storage and filming positions by pivoting one of the first and second segments 358, 360 about a pivot axis 364 relative to the other of the first and second segments 358, 360.

In the illustrated example, the first segment 358 includes a light mount aperture 394 and a microphone mount aperture 396. Referring to FIG. 15, in use, the light mount aperture 394 holds an external light 395 and the microphone mount aperture 396 holds an external microphone 397 to help film video with the portable electronic device 118. Preferably, when the device accessory 350 is in the filming position, the first and second segments 358 and 368 are inclined at an angle of about 90 degrees (e.g., see FIG. 15). Such an angle can allow the external light 395 and/or the external microphone 397 to be orientated so that they point in a similar direction as the device camera 135.

Referring to FIG. 14, when the device accessory 350 is in the storage position, the first and second segments 358, 360 extend generally parallel to one another (i.e., the first and second segments 358, 360 extend in the same plane). The accessory mount 100 (FIG. 2) can be stowed within the device accessory 350 when the device accessory 250 is in the storage position. Such a stowed arrangement can be space saving when the device accessory 350 is not being used.

Referring to FIG. 15, when the device accessory 350 is in the filming position, the first segment 358 is inclined relative to the second segment 360. In the illustrated example, the first segment 358 is positioned above the portable electronic device 118 and the light 395 and microphone 397 are pointed in the same direction as the device camera 135.

In the illustrated example, the second segment 360 includes a device accessibility aperture 392. Referring to FIG. 15, when the device accessory 350 is in the filming position, the device accessibility aperture 392 overlies a corresponding device accessibility aperture 136 on the accessory mount 100 to avert obstruction of the device camera 135.

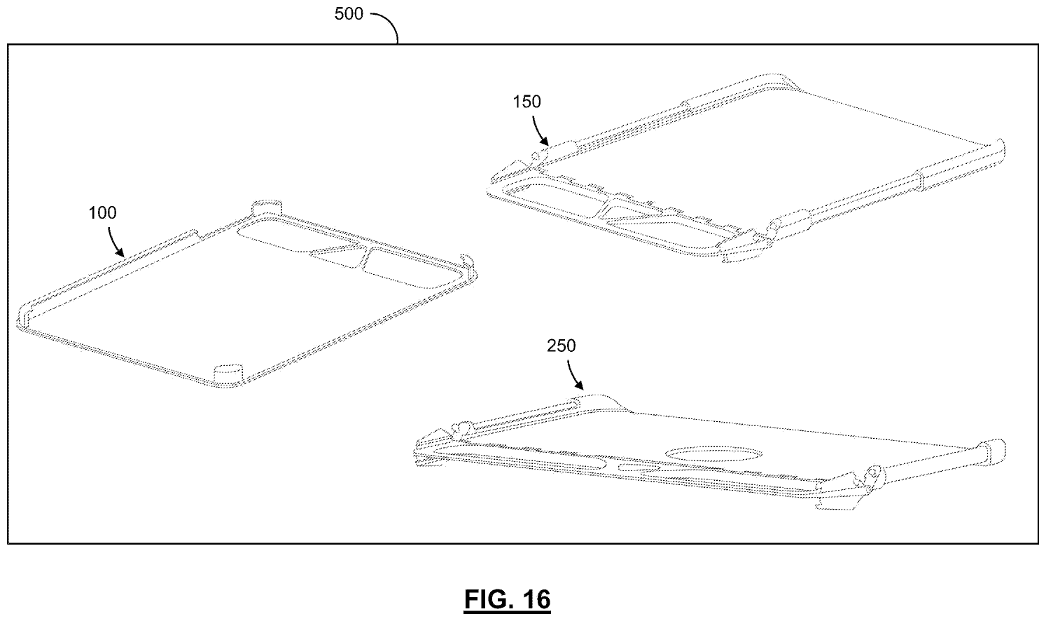

Referring now to FIG. 16, an accessory kit 500 includes the accessory mount 100, the device accessory 150 and the device accessory 250. In some examples, the kit 500 includes only one device accessory. As an example, the kit 500 may include the accessory mount 100 and either the device accessory 150 or the device accessory 250. As another example, the kit 500 may include the accessory mount 100 and the device accessory 350. In some examples, the kit 500 may include more than two device accessories. As an example, the kit may include the accessory mount 100, the device accessory 150, the device accessory 250 and the device accessory 350.

The accessory kit 500 optionally includes one or more user item holders 300, 1300. For example, the kit 500 may include a plurality of different user item holders (e.g., 2-20). This would allow a user to select and connect one of the plurality of user item holders to the device accessory 150, 250 according to their needs. Although the example user item holders 300, 1300 described and illustrated herein are releasably securable to the device accessory 150, user item holders that are releasably securable to the device accessory 250 are fully contemplated and omitted from the drawings solely for brevity.

What has been described above is intended to be illustrative of examples of the teaching disclosed herein, without limiting the scope of patent claims granted herefrom. The scope of such claims should be given the broadest interpretation consistent with the description as a whole.

1. An accessory mount for a portable electronic device, the accessory mount comprising:
a mount body having longitudinally opposed first and second sides and laterally opposed third and fourth sides;
a device fastener affixed to the mount body for releasably securing the accessory mount to the portable electronic device;
a first accessory attachment member extending from and about the first side of the mount body;
a second accessory attachment member extending from and about the second side of the mount body;
a third accessory attachment member extending from and about the third side of the mount body; and
a fourth accessory attachment member extending from and about the fourth side of the mount body,
wherein, the first and second accessory attachment members are spaced longitudinally apart by a first distance for slidable engagement with a first electronic device accessory, the third and fourth accessory attachment members are spaced laterally apart by a second distance for slidable engagement with a second electronic device accessory, and the first distance is greater than the second distance.

2. The accessory mount of claim 1, wherein the device fastener includes a sidewall extending generally perpendicularly from a periphery of the mount body and a device retaining ledge extending inboard from an upper edge of the sidewall.

3. The accessory mount of claim 2, wherein at least a portion of the mount body, the sidewall, and the retaining ledge define a recess for receiving a peripheral portion of the electronic device.

4. The accessory mount of claim 2, wherein each of the first, second, third and fourth accessory attachment members extends outboard of the sidewall.

5. The accessory mount of claim 2, wherein the device fastener comprises a plurality of fastener segments, each fastener segment being spaced apart from an adjacent one of the plurality of fastener segments along the periphery of the mount body, each fastener segment including a portion of the sidewall and a portion of the device retaining ledge.

6. The accessory mount of claim 1, wherein each of the first, second, third and fourth accessory attachment members extends respectively along a full length of the first, second, third, and fourth sides of the mount body.

7. The accessory mount of claim 1, further comprising an uninterrupted peripheral attachment lip extending about a perimeter of the mount body, the peripheral attachment lip including the first, second, third and fourth attachment members.

8. The accessory mount of claim 1, wherein the mount body includes a front surface and a rear surface opposite the front surface, and the front surface of the mount body at least partially overlies a rear surface of the portable electronic device when the accessory mount is secured to the portable electronic device, and wherein the first, second, third and fourth accessory attachment members are flush with the rear surface of the mount body.

9. The accessory mount of claim 1, wherein the mount body includes a mount magnet for magnetically engaging a device magnet of the portable electronic device to strengthen securement between the accessory mount and the portable electronic device.

10. An accessory kit for a portable electronic device, the accessory kit comprising:
a) an accessory mount including:
a mount body having longitudinally opposed first and second sides and laterally opposed third and fourth sides;
at least one device fastener affixed to the mount body for releasably securing the accessory mount to the portable electronic device;
a first accessory attachment member extending from and about the first side of the mount body;
a second accessory attachment member extending from and about the second side of the mount body;
a third accessory attachment member extending from and about the third side of the mount body; and
a fourth accessory attachment member extending from and about the fourth side of the mount body,
wherein, the first and second accessory attachment members are spaced longitudinally apart by a first distance, the third and fourth accessory attachment members are spaced laterally apart by a second distance, and the first distance is greater than the second distance; and
b) at least one device accessory slidably engageable with the accessory mount via one of i) the first and second accessory attachment members and ii) the third and fourth accessory attachment members.

11. The accessory kit of claim 10, wherein the at least one device accessory includes a first device accessory and a second device accessory, the first device accessory slidably engageable with the accessory mount via the first and second accessory attachment members, and the second device accessory slidably engageable with the accessory mount via the third and fourth accessory attachment members.

12. The accessory kit of claim 10, wherein each device accessory includes a pair of opposed mounting members spaced apart by one of the first distance and the second distance, and
when the mounting members are spaced apart by the first distance, each mounting member is configured to slidably engage a respective one of the first and second accessory attachment members, and
when the mounting members are spaced apart by the second distance, each mounting member is configured to slidably engage a respective one of the third and fourth accessory attachment members.

13. The accessory kit of claim 12, wherein each device accessory includes a pair of spaced apart accessory sidewalls, and each mounting member is formed as a channel extending along a respective one of the accessory sidewalls.

14. The accessory kit of claim 13, wherein each device accessory includes a first segment and a second segment pivotably connected to an edge of the first segment, and each device accessory is movable between a storage position, in which the first and second segments extend generally parallel to one another, and at least a device standing position, in which the first segment is inclined relative to the second segment by a stand angle.

15. An accessory assembly for a portable electronic device, the accessory assembly comprising:
a) an accessory mount including:
a mount body;
at least one device fastener affixed to the mount body for releasably securing the accessory mount to the portable electronic device;
a pair of opposed accessory attachment members including a first accessory attachment member extending from and about a first side of the mount body and a second accessory attachment member extending from and about a second side of the mount body opposite the first side of the mount body;
b) a device accessory slidably engageable with the accessory mount, the device accessory including:
a pair of opposed mounting members, each mounting member being configured to slidably engage a respective one of the first and second accessory attachment members of the accessory mount;
a first segment and a second segment pivotably connected to an edge of the first segment, the device accessory being movable between a storage position, in which the first and second segments extend generally parallel to one another, and at least a device standing position, in which the second segment is inclined relative to the first segment by a stand angle; and
c) a user item holder releasably securable to the second segment, the user item holder including a pair of opposed clips for engaging opposed edges of the second segment to secure the user item holder to the second segment.

16. The accessory assembly of claim 15, wherein the user item holder includes a holder body having opposed first and second holder body edges, the pair of opposed clips includes a first clip extending generally perpendicularly from the first holder body edge and a second clip extending generally perpendicular from the second holder body edge.

17. The accessory assembly of claim 16, wherein the second segment has a length between the opposed edges engageable with respective ones of the first and second clips, and wherein the first and second clips are spaced apart by a clip gap sized to accommodate the length of the second segment.

18. The accessory assembly of claim 16, wherein the holder body has an outer surface, and the accessory assembly further comprises a storage compartment affixed to the outer surface of the holder body and configured to hold a user item.

19. The accessory assembly of claim 18, wherein the holder body has an inner surface opposite the outer surface, and when the user item holder is secured to the second segment, the inner surface of the holder body overlies at least a portion of an outer surface of the second segment.

20. The accessory assembly of claim 19, wherein
the holder body has opposed third and fourth holder body edges that extend generally perpendicular to the first and second holder body edges, the third and fourth holder body edges being spaced apart by a holder body depth, and
the outer surface of the second segment includes a first retaining ridge and a second retaining ridge spaced apart from and extending parallel to the first retaining ridge, the first and second retaining ridges being spaced apart by a ridge gap sized to accommodate the holder body depth and prevent sliding of the user item holder when secured to the second segment.

21. The accessory assembly of claim 20, wherein the outer surface of the second segment has a recessed portion between the first and second retaining ridges, and when the user item holder is secured to the second segment, the holder body is seated in the recessed portion.
